(19) United States
(12) Patent Application Publication
(10) Pub. No.: US 2009/0118211 A1
(43) Pub. Date: May 7, 2009

(54) Compositions and Methods for Enhancement of Sexual Function

(76) Inventors: Daniel Drai, Tel-Aviv (IL); Sivan Goren, Givataim (IL)

Correspondence Address: MARTIN D. MOYNIHAN d/b/a PRTSI, INC., P.O. BOX 16446, ARLINGTON, VA, 22215, US

(21) Appl. No.: 11/922,860

(22) PCT Filed: Jun. 27, 2006
(86) PCT No.: PCT/IL2006/000747

§ 371 Date: Dec. 26, 2007

Related U.S. Application Data
(60) Provisional application No. 60/693,798, filed on Jun. 27, 2005.
(60) Provisional application No. 60/708,764, filed on Aug. 17, 2005.

(51) Int. Cl. A61K 31/7088; A61K 31/55; A61K 31/4985; A61K 31/519; A61P 15/00

(52) U.S. Cl. 514/44; 514/217; 514/250; 514/262.1

(57) ABSTRACT

Novel compositions and methods which utilize an erection-enhancing agent and an ejaculation-delaying agent, such that these agents provide an overlapping and/or synergistic effect, are provided. These composition and methods can be beneficially utilized for treating sexual dysfunction and/or for enhancing sexual function.

### FIELD AND BACKGROUND OF THE INVENTION

The present invention relates to compositions and methods for treating sexual dysfunctions and/or enhancing sexual function.

Sexual dysfunctions may begin early in a person's life, or they may develop after an individual has previously experienced enjoyable and satisfying sex. A problem may develop gradually over time, or may occur suddenly as a total or partial inability to participate in one or more stages of the sexual act.

The causes of sexual dysfunctions can be physical, psychological, or both. Psychological factors include both interpersonal problems and psychological problems within the individual, such as depression.

Physical factors contributing to sexual dysfunctions include drugs (alcohol, nicotine, narcotics, stimulants, antihypertensives, antihistamines, and some psychotherapeutic drugs); injuries to the back; problems with an enlarged prostate gland; problems with blood supply; nerve damage (as in spinal cord injuries); disease (diabetic neuropathy, multiple sclerosis, tumors, and, rarely, tertiary syphilis); failure of various organ systems (such as the heart and lungs); endocrine disorders (thyroid, pituitary, or adrenal gland problems); hormonal deficiencies (low testosterone, estrogen, or androgens); and some birth defects.

Sexual dysfunctions are generally classified into 4 categories: sexual desire disorders, sexual arousal disorders, orgasm disorders, and sexual pain disorders.

Sexual arousal disorders in men include erectile dysfunction, which involves partial or complete failure to attain or maintain an erection adequately for intercourse. Erectile dysfunction is a very common problem, affecting from about 40% to 60% of men at some time in their life, and about 52% of men between 40 to 70 years old [1]. However, only about 10% of men suffering from erectile dysfunction are willing to seek medical help.

Patients suffering from erectile dysfunction generally respond well to medication of the phosphodiesterase type 5 (PDE5) family. For example, 81% of subjects studied were found to respond well to oral administration of 20 mg of tadalafil (Cialis™) [2], and between 74% to 91% were found to respond well to oral administration of 20 mg of vardenafil (Levitra™) [4] and 82% were found to respond well to oral administration of 50-100 mg of sildenafil (Viagra™).

In addition to erectile dysfunction patients, it has been found that many consumers of PDE5 are physically healthy men, with no pathological sexual problems, who are aiming to improve the quality of their sexual performance by enhancing extent and duration of erection.

The principal drugs belonging to the PDE5 family are tadalafil (Cialis™), vardenafil (Levitra™) and sildenafil (Viagra™).

It has been shown that about 79% of patients treated with tadalafil were able to have sexual intercourse with a satisfactory erection [1] within about 2 to 36 hours after administration.

Pharmacokinetic studies have shown that tadalafil (20 mg) is absorbed after oral administration, with a maximum observed plasma concentration (Cₘₐₓ ) of 378 μg/L and a time to reach maximum plasma concentration (Tₘₐₓ) of 2 hours (20 mg dose) [1, 2]. Systemic exposure (AUC) increases proportionately with doses from 2.5 mg to 20 mg. The increase in Cₘₐₓ is slightly less than proportional over that dose range. During once-daily 20 mg dosing, steady-state plasma concentrations were attained within approximately 5 days and the degree of drug accumulation was 1.6 fold.

Pharmacokinetic studies have shown that sildenafil (50-100 mg) is absorbed after oral administration, with a maximum observed plasma concentration (Cₘₐₓ) of 18 ng/ml and a time to reach maximum plasma concentration (Tₘₐₓ) of 60 minutes (50-100 mg dose) [1, 2].

Studies with vardenafil have been shown that about 82% of subjects studied can have sexual intercourse with a satisfactory erection [3] within 25 minutes to 5 hours after an administrated dose of 20 mg vardenafil. Vardenafil is rapidly absorbed after oral administration, with Cₘₐₓ being reached as early as 15 minutes post-administration. After oral dosing of vardenafil, AUC and Cₘₐₓ increase in almost direct proportion to the dose over the recommended dose range (5-20 mg). 82% of subjects studied can have sexual intercourse with a satisfactory erection within 14 minutes to 6 hours after an administered dose of 50-100 mg vardenafil.

Ejaculatory dysfunctions include delayed ejaculation and premature ejaculation. Premature ejaculation is the most common and underestimated male sexual disorder.

Premature ejaculation is a frequent and distinct medical condition, affecting from 20-60% of men worldwide, which can severely impact quality of life, affecting the physical and emotional well-being of patients and their partners. More common than erectile dysfunction, this condition can affect men at any point in their lives, and one in four men experience poor control over ejaculation on a frequent basis.

Men with poor control over ejaculation tend to be less satisfied with sexual intercourse and their sexual relationship, and may suffer more difficulties with sexual anxiety and arousal compared to non-sufferer. In one study [6], men classified with probable premature ejaculation self-reported poor control over ejaculation, low satisfaction with sexual intercourse, low satisfaction with sexual relationship, low interest in actually having sexual intercourse, difficulty in becoming sexually aroused, and difficulty relaxing during intercourse. These findings highlight the negative impact of premature ejaculation on quality of life, sexual performance and enjoyment of sex.

Based on psychopharmacological studies there is evidence that premature ejaculation is related to a diminished serotonergic neurotransmission, and 5-HT2C or 5-HT1A receptor disturbances. Moreover, animal studies show the presence of a distinct ejaculation-related neural circuit in the central nervous system [7].

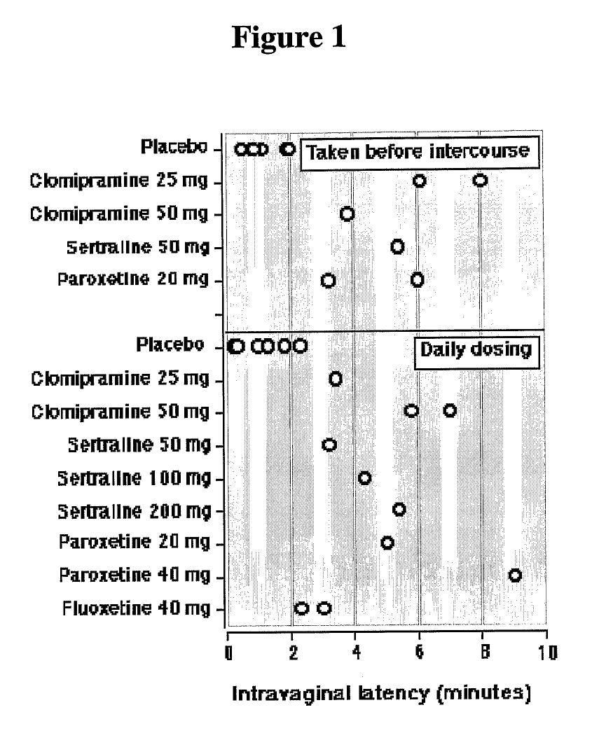

Some psychoactive drugs, particularly tricyclic antidepressants, such as clomipramine, and selective serotonin reuptake inhibitors (SSRIs) such as fluoxetine, paroxetine and sertraline, can delay ejaculation as a side effect. Almost all members of the SSRI family have been shown to be able to delay ejaculation, though to various extents. FIG. 1 presents representative results, described in published studies [15, 16], showing the effect of various antidepressant and placebo on the ejaculatory latency time of men.

Clomipramine is a chlorinated analogue of imipramine [1] with both antidepressant and antiobsessional properties [2, 3], which penetrates the blood-brain barrier readily, reaching concentrations in the brain that are ten times greater than plasma concentrations after a single parenteral dose [4, 7].

Clomipramine inhibits norepinephrine and serotonin uptake into central nerve terminals, possibly by blocking the membrane-pump of neurons, thereby increasing the concentration of transmitter monoamines at receptor sites. Clomipramine also possesses anticholinergic properties, weak antihistamine and antiserotonin properties, potentiates the effect of norepinephrine and other drugs acting on the central nervous system, has a quinidine-like effect on the heart and may impair cardiac conduction.

Clomipramine has been shown to block ejaculation without suppressing orgasm [8, 9]. Clomipramine has an effect on the excretory phase of ejaculation, by inhibiting contraction of the longitudinal fibers of the deferent ducts responsible for the sperm progression, without affecting the contractile activity of their circular fibers which act as a clamp, blocking the passage of the semen [17].

A significant increase in time to ejaculation has been demonstrated in men who took 25 milligrams of clomipramine 12 to 24 hours before anticipated sexual activity. The latency to ejaculation ranged from about 1.5 to 2 minutes up to about 8 minutes, which was considered to be in the range in which sexual satisfaction was reported [10].

Another study reported that 50 mg of clomipramine can extend time to climax in premature ejaculators to more than 8 minutes [11, 12]. The researcher also noted that the advantage of taking only a small dose of clomipramine, only as needed, helps reduce the number of side effects that often occur when people take antidepressants over long periods of time.

Plasma levels of clomipramine usually peak 2-6 hours after dosage but much individual variation occurs. The half-life of clomipramine ranges from 8.6 to 36 hours.

Premature ejaculation is frequently associated with erectile dysfunction. However, clomipramine alone has been shown to be ineffective in men with premature ejaculations who were also impotent [10], while sildenafil and PDE5 alone was ineffective for men with premature ejaculation. The administration of PDE5 may even aggravate premature ejaculation.

Furthermore, healthy subjects frequently seek to improve the quality of their sexual relationships, by both strengthening erection and delaying time to ejaculation, in order to experience a longer and more intense orgasm and provide their partner with increased sexual pleasure. For such subjects, administration of an erection enhancing agent and an ejaculation-delaying agent would be desirable.

The use of PDE5 drugs allows for lengthier foreplay, which increases female desire but also leads to stronger arousal for the male partner, thus presenting a higher risk of premature ejaculation, or at least ejaculation before the desired time. Therefore, these subjects may benefit from co-administration of an ejaculation-delaying agent.

Men suffering from performance anxiety leading to erectile dysfunction or loss of erection during foreplay or penetration frequently have problems with control of ejaculation, which will often be premature. There is therefore a real need for a solution to the problem of premature ejaculation combined with erectile dysfunction.

Furthermore, women suffering from difficulty in reaching climax or from pain during penetration need a partner who can control his ejaculation without losing his erection in order to enable her to relax, without fearing a too rapid ejaculation. In this case also, there is a need to enable the healthy partner to delay his ejaculation without losing his erection, thus contributing to the treatment and improvement of his partner's sexuality.

The use of the SSRI drug paroxetine together with the phosphodiesterase type 5 inhibitor sildenafil, has been reported for treatment of potent patients with premature ejaculation [13]. In this study, patients received either paroxetine alone, 3-4 hours before sexual intercourse, or a combined therapy of paroxetine, 3-4 hours before sexual intercourse, and sildenafil, 1 hour before sexual intercourse, while evaluating the effect of the addition of sildenafil on premature ejaculation.

In addition, the effect of SSRIs in the treatment of premature ejaculation in patients treated with sildenafil for erectile enhancement has been reported [14]. In this study, the effect of the SSRI sertraline on premature ejaculation in patients having primary premature ejaculation was compared to its effect in patients having sildenafil-corrected erectile dysfunction. All patients received sertraline 4 hours before sex. Sertraline was found effective as ejaculation delaying agent only for patients having primary premature ejaculation.

Prolonged treatment with SSRIs, however, has been shown to adversely affect sexual dysfunction. Between 30% and 50% of SSRI-treated patients experience sexual dysfunction. Several studies, aiming at reducing SSRI-induced sexual dysfunction have been published. Thus, for example, it was reported that administration of ropinrole, an anti-parkinsonian dopamine agonist, was effective in treating sexual dysfunction induced by citalopram, sertraline, paroxetine, fluoxetine and venlafaxine treatment [Ginzberg D. L., Primary Psychiatry, 2003:10(2), 22-26]. U.S. Patent Application Publication No. 2003/0055070 teaches the use of PDE5 inhibitors for treating SSRI-induced sexual dysfunction. None of these publications, however, addresses a combined treatment of erectile dysfunction and premature ejaculation.

U.S. Patent Application Publication No. 2005/0009835 teaches a combined therapy that comprises co-administration and/or co-formulation of a PDE5 inhibitor and 1-deprenyl or propargylamine compounds, the latter being monoamine oxidase inhibitors that enhance dopamine activity and increase cGMP generation, and thereby may augment the action of the PDE5 inhibitor in treating sexual dysfunction. This patent application, however, fails to teach a combined therapy that addresses both erectile dysfunction and premature ejaculation.

WO 02/079152 discloses a novel SSRI compound which was found effective in treating, inter alia, premature ejaculation. While this patent application teaches the use of this compound, in combination with an erection dysfunction agent such as sildenafil, for treating sexual dysfunction, the effect of such a combined therapy has not been demonstrated.

WO 02/41883 and U.S. Pat. Nos. 6,495,154 and 6,946,141 teach a method of treating premature ejaculation, which is effected by administration of a cyclic (e.g., tricyclic, tetracyclic and other) non-SRI antidepressant such as clomipramine, on an “as needed” basis, which allows administration of the active agent shortly (within a time frame of 0.25-3.5 hours) before anticipated sexual activity. According to the teachings of this patent application, an additional active agent such as a PDE5 inhibitor can be co-administered together with the active agent. The teachings of these patents and patent application are based on a rapid-release pharmaceutical formulation. This patent application, however, fails to teach a method that is directed at simultaneously providing an ejaculation delaying effect and an erection enhancing effect.

Thus, while the prior art teaches various methods that involve co-administration of an erection enhancement agent and anti-depressants such as SSRIs and tricyclic anti-depressants, the prior art does not teach or suggest co-administration of an erection-enhancing agent and an ejaculation-delaying agent for the treatment of patients suffering from erectile dysfunction. The prior art also does not teach or suggest co-administration of the two agents to healthy subjects seeking to enhance erection and delay ejaculation.

Furthermore, the known agents for enhancement of erection and delay of ejaculation involve drugs which reach maximum plasma concentrations at different times, requiring administration of the individual medications at separate times, which is highly inconvenient for the patient. The prior art does not teach or suggest a treatment which provides substantially simultaneous co-administration of an agent for enhancement of erection and an agent for delay of ejaculation. The prior art further does not teach or suggest co-formulation of an agent for enhancement of erection and an agent for delay of ejaculation and particularly fail to teach such a co-formulation which provides an overlapping maximal efficacy of these agents.

There is thus a widely recognized need for and it would be highly advantageous to have, compositions and methods for enhancement of sexual function, devoid of the above limitations.

### SUMMARY OF THE INVENTION

The present invention successfully addresses the shortcomings of the presently known methods of enhancing sexual function by providing methods and compositions for co-administration of an erection-enhancing agent and an ejaculation-delaying agent which provide an overlapping and/or synergistic effect of these agents and thus can be efficiently utilized in the treatment of patients suffering from sexual dysfunction, premature ejaculation and/or erectile dysfunction.

Thus, according to one aspect of the present invention there is provided a pharmaceutical composition comprising a pharmaceutically effective amount of an erection-enhancing agent and a pharmaceutically effective amount of an ejaculation-delaying agent, the composition being such that an efficacy window of the an erection-enhancing agent and an efficacy window of the ejaculation-delaying agent substantially overlap.

According to another aspect of the present invention there is provided a pharmaceutical composition comprising a pharmaceutically effective amount of an erection-enhancing agent and a pharmaceutically effective amount of an ejaculation-delaying agent, the composition being such that the erection-enhancing agent and the ejaculation-delaying agent act in synergy. The pharmaceutically effective amount of the erection-enhancing agent therefore preferably equals to or is lower than 25 mg per dosage unit, whereby the pharmaceutically effective amount of the ejaculation-delaying agent is preferably lower than 50 mg per dosage unit.

According to still another aspect of the present invention there is provided a pharmaceutical composition comprising clomipramine, as an ejaculation-delaying agent, and a PDE5 inhibitor, as an erection enhancing agent.

According to further features in preferred embodiments of the invention described below, the pharmaceutical compositions described herein are such that a maximal efficacy window of the erection-enhancing agent and a maximal efficacy window of the ejaculation-delaying agent overlap for at least 20 minutes.

According to still further features in the described preferred embodiments the pharmaceutical compositions described herein are designed such that a plasma peak concentration of the ejaculation-delaying agent and a plasma peak concentration of the erection-enhancing agent occur substantially simultaneously.

According to still further features in the described preferred embodiments the at least the erection-enhancing agent is provided in a delayed release form.

According to still further features in the described preferred embodiments the release of the erection-enhancing agent is delayed by from about 2 to about 3 hours relative to release of the ejaculation-delaying agent.

According to still further features in the described preferred embodiments the ejaculation-delaying agent is clomipramine.

According to still further features in the described preferred embodiments the erection-enhancing agent is selected from the group consisting of a phosphodiesterase type 5 inhibitor, arginine and human DNA.

According to still further features in the described preferred embodiments the phosphodiesterase type 5 inhibitor is selected from the group consisting of tadalafil, vardenafil and sildenafil.

According to still further features in the described preferred embodiments the pharmaceutical composition is formulated as a pharmaceutical dosage unit.

According to still further features in the described preferred embodiments an amount of the clomipramine ranges from about 15 mg to about 30 mg per dosage unit.

According to still further features in the described preferred embodiments the erection-enhancing agent comprises tadalafil.

According to still further features in the described preferred embodiments the lo amount of the tadalafil ranges from about 10 mg to about 20 mg per dosage unit and is preferably about 10 mg.

According to still further features in the described preferred embodiments the erection-enhancing agent comprises vardenafil.

According to still further features in the described preferred embodiments an amount the vardenafil ranges from about 10 mg to about 20 mg per dosage unit and is preferably about 10 mg.

According to still further features in the described preferred embodiments the erection-enhancing agent comprises sildenafil.

According to still further features in the described preferred embodiments an amount of the sildenafil is about 25 mg per dosage unit.

According to still further features in the described preferred embodiments a release of the tadalafil is delayed by about 2 hours relative to a release of the clomipramine.

According to still further features in the described preferred embodiments a release of the vardenafil is delayed by about 3 hours relative to a release of the clomipramine.

According to still further features in the described preferred embodiments the release of the sildenafil is delayed by about 3 hours relative to a release of the clomipramine.

According to still further features in the described preferred embodiments the pharmaceutical compositions described herein are provided in a delivery form suitable for oral administration.

According to still further features in the described preferred embodiments the pharmaceutical compositions described herein further comprise a pharmaceutically acceptable carrier.

According to an additional aspect of the present invention there is provided a method of enhancing sexual satisfaction, the method comprising co-administering a pharmaceutically effective amount of an erection-enhancing agent and a pharmaceutically effective amount of an ejaculation-delaying agent, such that an efficacy window of the erection-enhancing agent and an efficacy window of the ejaculation-delaying agent substantially overlap, thereby enhancing sexual dysfunction.

According to still an additional aspect of the present invention there is provided a method of enhancing sexual satisfaction, the method comprising co-administering a pharmaceutically effective amount of an erection-enhancing agent and a pharmaceutically effective amount of an ejaculation-delaying agent, the erection-enhancing agent and the ejaculation-delaying agent acting in synergy, thereby enhancing sexual dysfunction.

According to yet an additional aspect of the present invention there is provided a use of an erection-enhancing agent and an ejaculation-delaying agent, wherein an efficacy window of the erection-enhancing agent and an efficacy window of the ejaculation-delaying agent substantially overlap, for the preparation of a medicament for enhancing sexual satisfaction.

According to a further aspect of the present invention there is provided a use of an erection-enhancing agent and an ejaculation-delaying agent, wherein the erection-enhancing agent and the ejaculation-delaying acting in synergy, for the preparation of a medicament for enhancing sexual satisfaction.

According to still a further aspect of the present invention there is provided a method of treating erectile dysfunction, the method comprising co-administering to a subject in need thereof a pharmaceutically effective amount of an erection-enhancing agent and a pharmaceutically effective amount of an ejaculation-delaying agent, such that an efficacy window of the an erection-enhancing agent and an efficacy window of the ejaculation-delaying agent substantially overlap, thereby treating erectile dysfunction.

According to yet a further aspect of the present invention there is provided a method of treating erectile dysfunction, the method comprising co-administering to a subject in need thereof a pharmaceutically effective amount of an erection-enhancing agent and a pharmaceutically effective amount of an ejaculation-delaying agent, the erection-enhancing agent and the ejaculation-delaying agent acting in synergy, thereby treating erectile dysfunction.

According to another aspect of the present invention there is provided a method of treating erectile dysfunction, the method comprising co-administering to a subject in need thereof a pharmaceutically effective amount of a phosphodiesterase type 5 inhibitor, as an erection-enhancing agent and a pharmaceutically effective amount of clomipramine, as an ejaculation-delaying agent, thereby treating erectile dysfunction.

According to still another aspect of the present invention there is provided a use of an erection-enhancing agent and an ejaculation-delaying agent, wherein an efficacy window of the erection-enhancing agent and an efficacy window of the ejaculation-delaying agent substantially overlap, for the preparation of a medicament for treating erectile dysfunction.

According to yet another aspect of the present invention there is provided a use of an erection-enhancing agent and an ejaculation-delaying agent, wherein the erection-enhancing agent and the ejaculation-delaying agent act in synergy, for the preparation of a medicament for treating erectile dysfunction.

According to still another aspect of the present invention there is provided a use of a phosphodiesterase type 5 inhibitor as an erection-enhancing agent and clomipramine as an ejaculation-delaying agent, for the preparation of a medicament for treating erectile dysfunction.

According to an additional aspect of the present invention there is provided a method of treating sexual dysfunction, the method comprising substantially simultaneously co-administering to a subject in need thereof a pharmaceutically effective amount of an erection-enhancing agent and a pharmaceutically effective amount of an ejaculation-delaying agent, such that an efficacy window of the an erection-enhancing agent and an efficacy window of the ejaculation-delaying agent substantially overlap.

According to still an additional aspect of the present invention there is provided a method of treating sexual dysfunction, the method comprising substantially simultaneously co-administering to a subject in need thereof a pharmaceutically effective amount of an erection-enhancing agent and a pharmaceutically effective amount of an ejaculation-delaying agent, the an erection-enhancing agent and the ejaculation-delaying agent acting in synergy, thereby treating sexual dysfunction.

According to yet an additional aspect of the present invention there is provided a method of treating sexual dysfunction, the method comprising co-administering to a subject in need thereof a pharmaceutically effective amount of a phosphodiesterase type 5 inhibitor as an erection-enhancing agent and a pharmaceutically effective amount of clomipramine as an ejaculation-delaying agent.

Preferably, the co-administering is performed substantially simultaneously.

According to a further aspect of the present invention there is provided a use of an erection-enhancing agent and an ejaculation-delaying agent, wherein an efficacy window of the erection-enhancing agent and an efficacy window of the ejaculation-delaying agent substantially overlap, for the preparation of a medicament for treating sexual dysfunction.

According to still a further aspect of the present invention there is provided a use of an erection-enhancing agent and an ejaculation-delaying agent, wherein the erection-enhancing agent and the ejaculation-delaying agent acting in synergy, for the preparation of a medicament for treating sexual dysfunction.

According to yet a further aspect of the present invention there is provided a use of a phosphodiesterase type 5 inhibitor as an erection-enhancing agent and clomipramine as an ejaculation-delaying agent, for the preparation of a medicament for treating sexual dysfunction.

According to an additional aspect of the present invention there is provided a method of treating ejaculatory dysfunction caused by treatment with an agent for the treatment of erectile dysfunction, the method comprising substantially simultaneously co-administering to a subject in need thereof a pharmaceutically effective amount of an erection-enhancing agent and a pharmaceutically effective amount of an ejaculation-delaying agent, such that an efficacy window of the an erection-enhancing agent and an efficacy window of the ejaculation-delaying agent substantially overlap.

According to still an additional aspect of the present invention there is provided a use of an erection-enhancing agent and an ejaculation-delaying agent, wherein an efficacy window of the erection-enhancing agent and an efficacy window of the ejaculation-delaying agent substantially overlap, for the preparation of a medicament for treating ejaculatory dysfunction caused by treatment with an agent for the treatment of erectile dysfunction.

According to further features in preferred embodiments of the invention described below, the erectile dysfunction is caused by treatment with an ejaculation-delaying agent.

According to still further features in the described preferred embodiments the pharmaceutically effective amount of the erection-enhancing agent equals to or is lo lower than 20 mg per dosage unit.

According to still further features in the described preferred embodiments the pharmaceutically effective amount of the ejaculation-delaying agent is lower than 50 mg per dosage unit.

According to still further features in the described preferred embodiments at least the erection-enhancing agent is provided in a delayed release form.

According to still further features in the described preferred embodiments a release of the erection-enhancing agent is delayed by from about 2 to about 3 hours relative to a release of the erection-delaying agent.

According to still further features in the described preferred embodiments the ejaculation-delaying agent is clomipramine.

According to still further features in the described preferred embodiments the erection-enhancing agent is a phosphodiesterase type 5 inhibitor.

According to still further features in the described preferred embodiments the phosphodiesterase type 5 inhibitor is selected from the group consisting of tadalafil, vardenafil and sildenafil.

According to still further features in the described preferred embodiments the ejaculation-delaying agent is clomipramine.

According to still further features in the described preferred embodiments the clomipramine and the erection-enhancing agent are each provided as a pharmaceutical dosage unit.

According to still further features in the described preferred embodiments an amount of the clomipramine ranges from about 15 mg to about 30 mg per dosage unit.

According to still further features in the described preferred embodiments the erection-enhancing agent comprises tadalafil.

According to still further features in the described preferred embodiments an amount of the tadalafil ranges from about 10 mg to about 20 mg per dosage unit.

According to still further features in the described preferred embodiments an amount of the tadalafil ranges is about 10 mg per dosage unit.

According to still further features in the described preferred embodiments the erection-enhancing agent comprises vardenafil.

According to still further features in the described preferred embodiments an lo amount of the vardenafil ranges from about 10 mg to about 20 mg per dosage unit.

According to still further features in the described preferred embodiments an amount of the vardenafil ranges is about 10 mg per dosage unit.

According to still further features in the described preferred embodiments the erection-enhancing agent comprises sildenafil.

According to still further features in the described preferred embodiments an amount of the sildenafil is about 25 mg per dosage unit.

According to still further features in the described preferred embodiments a release of the tadalafil is delayed by about 2 hours relative to a release of the clomipramine.

According to still further features in the described preferred embodiments a release of the vardenafil is delayed by about 3 hours relative to a release of the clomipramine.

According to still further features in the described preferred embodiments a release of the sildenafil is delayed by about 3 hours relative to a release of the clomipramine.

According to still further features in the described preferred embodiments the administering is effected orally.

According to still further features in the described preferred embodiments the dysfunction is due to drug use.

According to still further features in the described preferred embodiments the drug is selected from the group consisting of alcohol, nicotine, a narcotic, a stimulant, an antihypertensive, an antihistamine, and a psychotherapeutic agent.

According to still further features in the described preferred embodiments the dysfunction is due to a medical condition.

According to still further features in the described preferred embodiments the medical condition is selected from the group consisting of a back injury, an enlarged prostate gland, a circulatory problem, nerve damage, diabetic neuropathy, multiple

FIG. 1 presents background art results showing the intravaginal latency times in patients treated with placebo or various antidepressants administered before sexual activity (upper part) and upon daily dosing (lower part) [15, 16]. Each point represents the intravaginal latency time for drug or placebo in a single arm of a randomized trial.

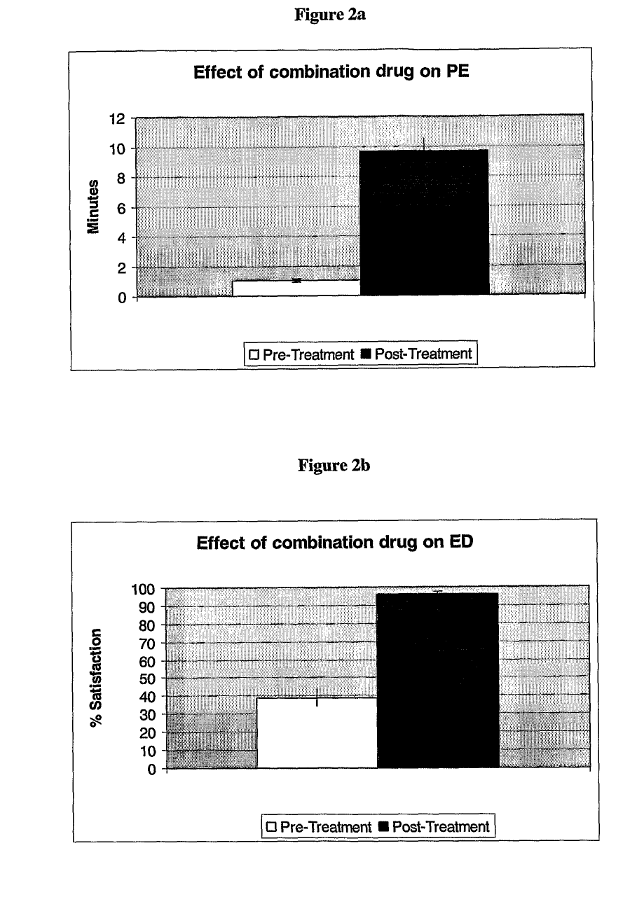

FIGS. 2a-b are bar graphs presenting the effect of a combined therapy of clomipramine and a PDE5 inhibitor, according to preferred embodiments of the present invention, on premature ejaculation (FIG. 2a) and on erection dysfunction (FIG. 2b).

### DESCRIPTION OF THE PREFERRED EMBODIMENTS

The present invention is of novel compositions and methods which utilize an erection-enhancing agent and an ejaculation-delaying agent, such that the efficacy windows of these agents substantially overlap and/or which act in synergy, and hence can be beneficially used for treating sexual dysfunction and/or for enhancing sexual function.

The principles and operation of the compositions and methods according to the present invention may be better understood with reference to the accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Although the prior art teaches agents for enhancement of erection and agents for delaying ejaculation, it does not teach or suggest a pharmaceutical composition comprising an erection-enhancing agent and an ejaculation-delaying agent or co-administration of an erection-enhancing agent and an ejaculation-delaying agent, in order to increase sexual satisfaction or treat erectile dysfunction. More specifically, the prior art fails to teach or suggest co-formulation and/or co-administration of an erection-enhancing agent and an ejaculation-delaying agent that would act so as to simultaneously exert an erection-enhancing affect and an ejaculation-delaying effect during a sexual intercourse.

Known methods of co-administration require administration of the individual medications at separate times, which is highly inconvenient for the patient, thereby reducing patient compliance. The prior art does not teach or suggest a method of substantially simultaneous co-administration of such agents. The prior art further fails to teach or suggest a combined therapy of an erection-enhancing agent and an ejaculation-delaying agent which act in synergy, particularly with regard to erection enhancement.

The present invention overcomes the deficiencies of the prior art by providing novel formulations which enable co-administration of an erection-enhancing agent and an ejaculation-delaying agent such that an erection-enhancement effect and an ejaculation-delay effect are exhibited simultaneously and/or synergistically.

Such formulations may comprise either a unitary dosage form, comprising an erection-enhancing agent and an ejaculation-delaying agent, or at least two individual dosage forms, each comprising one of an erection-enhancing agent and an ejaculation-delaying agent.

As is demonstrated in the Examples section that follows, it has been found that a combined therapy of clomipramine as an exemplary ejaculation-delaying agent and a phosphodiesterase type 5 inhibitor as an exemplary erection-enhancing agent may be utilized for simultaneously treating erectile dysfunction and premature ejaculation, whereby such a combined therapy further provides for a synergistic effect of these two agents, particularly with regard to erection enhancement.

Thus, according to one aspect of the present invention, there are provided novel pharmaceutical compositions, each comprising an erection-enhancing agent and an ejaculation-delaying agent.

The pharmaceutical compositions disclosed herein are particularly beneficial for enhancing sexual function and/or treating sexual dysfunction, as is detailed herein.

As used herein, the phrase “sexual function” refers to any one or combination of features involved in various stages of the sexual act and encompasses, for example, sexual desire, erection, ejaculation, ejaculatory latency time, and/or orgasm.

As used herein the term “sexual dysfunction” refers to difficulty during any stage of the sexual act that prevents an individual from enjoying sexual activity. “Sexual dysfunction” encompasses decreased sexual desire, the inability to have or sustain a penile erection (erectile dysfunction), the inability to ejaculate, ejaculatory latency time (premature ejaculation) and/or the inability to experience orgasm.

As used herein, an “erectile dysfunction” refers to the inability of a male mammal to achieve and maintain penile erection for satisfactory sexual intercourse.

The phrase “premature ejaculation” as used herein intends a sexual dysfunction wherein a male experiences persistent or recurrent ejaculation with minimal sexual stimulation before, upon, or shortly after penetration.

The pharmaceutical composition includes a pharmaceutically effective amount of an erection-enhancing agent and a pharmaceutically effective amount of an ejaculation-delaying agent (also referred to herein collectively as “active ingredients” or “active agents”).

As used herein the phrase “erection-enhancing agent” refers to an agent which potentiates and/or prolongs genital blood flow, as is further detailed hereinbelow.

As used herein, the phrase “ejaculation-delaying agent” refers to an agent which extends the latency period between induction of erection and occurrence of ejaculation.

As used herein, the phrase “pharmaceutical composition” refers to either a single packaged pharmaceutical dose unit (further referred to herein as a “unitary dosage form”) that includes both active ingredients or two separate units (e.g. two dosage forms) each including a single active ingredient and both preferably further packaged into a single delivery device (e.g. capsule).

In one embodiment, the pharmaceutical composition described herein are designed such that the efficacy window of the erection-enhancing agent and the efficacy window of the ejaculation-delaying agent substantially overlap.

As used herein, the phrase “efficacy window” describes a time frame during which an active agent exhibits a desired pharmacological effect, herein an erection-enhancing effect or an ejaculation-delaying effect, upon administration. In other words, this phrase describes that time period at which the plasma concentration of an active agent is equal to or higher than a minimal pharmacologically effective concentration thereof.

As is well known in the art, an efficacy window of an agent depends on various factors such as systemic absorbance rate, the time required to reach a plasma peak concentration and/or clearance rate.

As described hereinabove, commonly used erection-enhancing agents and ejaculation-delaying agents display different efficacy windows. As is further discussed hereinabove, it is often desirable to treat subjects suffering from sexual dysfunction with both an erection-enhancing agent and an ejaculation-delaying agent. It is further often desirable to treat subject suffering from erectile dysfunction with both an erection-enhancing agent and an ejaculation-delaying agent. It is further often desirable to treat subject suffering from premature ejaculation with both an erection-enhancing agent and an ejaculation-delaying agent.

Such a combined treatment is desirable since it enables a men subject to experience both satisfactory penile erection and satisfactory intravaginal latency period prior to ejaculation, within the same sexual event. However, this desired outcome can only be achieved if both agents exhibit their effect within the same time frame.

Thus, the pharmaceutical compositions presented herein are designed such that a window efficacy of the erection-enhancing agent and a window efficacy of the ejaculation-delaying agent substantially overlap.

As used in the context of this and other aspects of the present invention, the phrase “substantially overlap” with respect to the efficacy windows of the active agents means that during a certain time period upon administration of the composition described herein, both the erection-enhancing agent and the ejaculation-delaying agent exhibit a desired pharmacological effect to some extent, namely, a plasma concentration of each agent is equal to or is higher than a minimum pharmacologically effective concentration of the agent. The efficacy windows of the active agents can overlap for, for example, 5 minutes, 10 minutes, 15 minutes, 20 minutes, 25 minutes, 30 minutes, 1 hour, 2 hours, 3 hours and even for longer time periods. The efficacy windows of the active agents can overlap such that during the overlapping period, both agents exhibit a maximal efficacy, such that one agent exhibits a maximal efficacy while the other agent exhibits a partial efficacy or such that both agents exhibit a partial efficacy.

Preferably, the efficacy windows of the active agents overlap for at least 20 minutes, so as to allow a subject to experience the overlapping pharmacological effects of both active agents during the sexual event.

Further preferably, the composition describes herein is designed such that it provides at least 20 minutes of overlapping maximal efficacy of the two active ingredients. Thus, preferably, the composition described herein is such that a maximal efficacy window of the erection-enhancing agent and the maximal efficacy window of the ejaculation-delaying agent overlap for at least 20 minutes.

As used herein, the phrase “maximal efficacy window” describes that time frame upon administration of the active agent during which the agent exhibits a maximal efficacy.

A maximal efficacy is typically related to the plasma peak concentration of an active agent.

Thus, further preferably, the composition of the present invention is designed such that a plasma peak concentration of each of the active ingredients occurs substantially simultaneously, namely, within the same time period upon administration.

One approach for achieving the above is to attenuate the release of erection-enhancing agent with respect to the release of the ejaculation-delaying agent. In order to achieve such staggered release, both the erection-enhancing agent and the ejaculation-delaying agent may be in a delayed release form of varying release profile, or the ejaculation-delaying agent may be in immediate release form and only the erection-enhancing agent in a delayed release form. In any case, release of the erection-enhancing agent is preferably delayed by from about 2 to about 3 hours relative to release of the ejaculation-delaying agent. Preferably, each of the erection-enhancing agent and the ejaculation-delaying agent are delivered as individual pulses, at spaced-apart time intervals. Hence, for example, release of the ejaculation-delaying agent may be substantially immediate, i.e. occurring within 1-2 hours of ingestion. This initial release may be followed by a time interval of between 2 and 3 hours during which substantially no drug is released from the dosage form, after which the erection-enhancing agent is then released.

The composition of the present invention may be administered according to any suitable dosage regime. For example, the composition may be administered on a daily basis, or only as required, on an “as needed” basis.

The composition of the present invention may be provided in any form known to the art.

Thus, the composition can be formulated as a unitary dose, i.e., a single packaged dose which includes both active ingredients, or it can be formulated as two separate dose units each separately packaged, although preferably such formulation is further packaged into a single delivery device (e.g. capsule), since one object of the present invention is to facilitate simultaneous co-administration of the two active ingredients.

The latter formulation is exemplified by capsules housing tablets or drug-containing beads or particles, a first portion of which comprise the erection-enhancing agent as active ingredient, and a second portion of which comprise the ejaculation-delaying agent as active ingredient, wherein each portion provides a different drug release profile. The capsule material may be either hard or soft, and as will be appreciated by those skilled in the art of pharmaceutical science, typically comprises a tasteless, easily administered and water soluble compound such as gelatin, starch or cellulose. A preferred capsule material is gelatin. The capsules are preferably sealed, such as with gelatin bands or the like.

A unitary dosage form may comprise a single tablet with the first and second dosage units each representing an integral and discrete layer thereof. For example, drug-containing particles or drug-containing beads can be compressed together into a single tablet using conventional tabletting means.

Delayed release, whether with regard to a composition comprising a unitary dosage form, or to methods involving administration of individual dosage forms, may be achieved by any method known in the art. As will be appreciated by those skilled in the art, a number of methods are available for preparing drug-containing tablets or other dosage units which provide a variety of drug release profiles. Such methods include coating a drug or drug-containing composition, increasing the drug's particle size, placing the drug within a matrix, and forming complexes of the drug with a suitable complexing agent. Delayed release may be provided, for example, by coating a drug or a drug-containing composition with a selected membrane coating material, typically although not necessarily a polymeric material.

To bring about the desired pulsatile release profile for a dosage form comprised of encapsulated portions of tablets, beads, or particles, the portions comprising the ejaculation-delaying agent are provided with little or no coating material, and those comprising the erection-enhancing agent are provided with some degree of coating material. Thus, for example, a first tablet, which releases drug substantially immediately, may have a total coating weight of less than about 10%, and the second tablet may have a total coating weight in the range of approximately 10% to 30%. The preferred coating weights for particular coating materials may be readily determined by those skilled in the art by evaluating individual release profiles for dosage units prepared with different quantities of various coating materials.

When a coating is used to provide delayed release dosage units, particularly preferred coating materials comprise bioerodible, gradually hydrolyzable and/or gradually water-soluble polymers. The “coating weight,” or relative amount of coating material per dosage unit, generally dictates the time interval between ingestion and drug release. Suitable membrane coating materials for effecting delayed release include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, cellulose ester-ether phthalate, hydroxypropylcellulose phthalate, alkali salts of cellulose acetate phthalate, alkaline earth salts of cellulose acetate phthalate, hydroxypropylmethyl cellulose hexahydrophthalate, cellulose acetate hexahydrophthalate, and carboxymethylcellulose sodium; acrylic acid polymers and copolymers preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac, ammoniated shellac, shellac-acetyl alcohol, and shellac n-butyl stearate.

Conventional coating procedures and equipment may be used to coat the dosage units, i.e., the drug-containing tablets, beads or particles. For example, a delayed release coating composition may be applied using a coating pan, an airless spray technique, fluidized bed coating equipment, or the like.

Alternatively, the delayed release dosage units, e.g, tablets or particles, may be formulated by dispersing the drug within a matrix of a suitable material such as an insoluble plastic, a hydrophilic polymer, or a fatty compound. The insoluble plastic matrices may be comprised of, for example, polyvinyl chloride or polyethylene. Hydrophilic polymers useful for providing a matrix for a delayed release dosage unit include, but are not limited to, those described above as suitable coating materials. Fatty compounds for use as a matrix material include, but are not limited to, waxes generally (e.g., carnauba wax) and glyceryl tristearate. Once the active ingredient is mixed with the matrix material, the mixture can be compressed into tablets or processed into individual drug-containing particles.

Further examples of compositions in which the erection-enhancing agent is delayed with respect to the ejaculation-delaying agent include a formulation in which the ejaculation-delaying agent is provided in an outer, immediate release layer, which is released as a first pulse, while the erection-enhancing agent is contained within a core which is separated from the outer layer by a film layer of an enteric coating. The enteric coating slowly dissolves after the delivery of the first pulse of drug allowing the release of the second pulse.

Pharmaceutical compositions suitable for use in context of the present invention include compositions wherein the active ingredients are contained in an amount effective to achieve the intended purpose. More specifically, a therapeutically effective amount or a pharmaceutically effective amount means an amount of an agent effective to prevent, alleviate or ameliorate symptoms of a condition.

Determination of a therapeutically effective amount is well within the capability of those skilled in the art, especially in light of the detailed disclosure provided herein.

For any agent used in the various aspects of the present invention, the therapeutically effective amount or dose can be estimated initially from activity assays in animals (e.g., humans). For example, a dose can be formulated to achieve a circulating concentration range that includes the IC₅₀ as determined by activity assays (e.g., the concentration of the agent, which achieves a half-maximal erection enhancement efficacy or ejaculation delaying efficacy). Such information can be used to more accurately determine useful doses and regiments.

Toxicity and therapeutic efficacy of the active agents described herein can be determined by standard pharmaceutical procedures in experimental subjects

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active agents which are sufficient to maintain the desired effects, termed the minimal effective concentration (MEC). Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

The amount of a composition to be administered will, of course, be dependent on the subject being treated, the severity of the affliction, the manner of administration, the judgment of the prescribing physician, etc.

In another embodiment of the present invention, the pharmaceutical compositions described herein are such that the erection-enhancing agent and the ejaculation-delaying agent act in synergy.

As is demonstrated in the Examples section that follows, it was surprisingly found that a combined therapy that utilizes an erection-enhancing agent and an ejaculation-delaying agent results in a synergistic effect, which allows to use relatively low doses of these agents, while achieving a treatment success rate higher than that obtained when each of these agents is utilized alone.

The synergistic activity of these agents has been particularly demonstrated with regard to erection enhancement. Thus, while utilizing relatively low doses of an erection-enhancing agent, in combination with an ejaculation-delaying agent, a significant improvement in erection of patients suffering from erectile dysfunction was observed. The synergistic effect of the combined treatment taught herein is particularly demonstrated when comparing the success rate of this combined treatment with the success rate of an erection-enhancing agent when utilized alone. Thus, as is detailed in the Examples section that follows, the success rate reported upon treatment with a relatively low-dose dosage regime of an exemplary erection-enhancing agent in patients having mild, moderate or severe erectile dysfunction was significantly improved when patients similarly diagnosed by mild, moderate or severe erectile dysfunction received similar treatment regime of an erection-enhancing agent, in combination with an ejaculation-delaying agent.

As used herein, the phrase “mild erectile dysfunction” describes a subjectively rated erection that ranges from about 60% to about 90% of the subject's normal (100%) erection.

As used herein, the phrase “moderate erectile dysfunction” describes a subjectively rated erection that ranges from about 30% to about 60% of the subject's normal (100%) erection.

As used herein, the phrase “severe erectile dysfunction” describes a subjectively rated erection that ranges from about 0% to about 30% of the subject's normal (100%) erection.

Hence, according to preferred embodiments of the present invention, a pharmaceutically effective amount of an erection-enhancing agent equals to or is lower than 25 mg per unit dosage. A pharmaceutically effective amount of an ejaculation-delaying agent is preferably lower than 50 mg per unit dosage.

Ejaculation-delaying agents suitable for use in the context of the present invention are preferably psychoactive drugs, more preferably antidepressants. Non-limiting examples of suitable antidepressants include a selective serotonin reuptake inhibitor (such as citalopram, escitalopram oxalate, fluoxetine, fluvoxamine, paroxetine, and sertraline); a tricyclic antidepressant (such as amitryptiline, amoxapin, carbamezipin, clomipramine, desipramine, doxepin, imipramine, nortryptiline, protryptiline, mirtazepine and trimipramine); a monoamine oxidase inhibitor (such as deprenyl, isocarboxazid, moclobemide, phenelzine and tranylcypromine); and a serotonin and noradrenaline uptake inhibitor (such as duloxetine, milnacipran and venlafaxine), or a combination thereof. Preferably, the ejaculation-delaying agent is a tricyclic antidepressant.

According to the presently most preferred embodiments of the present invention, the ejaculation-delaying agent is clomipramine.

Further according to preferred embodiments of the present invention, the erection-enhancing agent is a phosphodiesterase type 5 inhibitor, more preferably at least one of tadalafil, vardenafil and sildenafil.

Thus, preferred pharmaceutical compositions according to the present embodiments comprise clomipramine and at least one of tadalafil, vardenafil and sildenafil.

Alternatively, the erection-enhancing agent may be a natural product, such as arginine or human DNA.

Each of the active agents described herein can be utilized either as is or as a pharmaceutically acceptable salt, a solvate, or a hydrate thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the pharmacological activity and properties of the administered compound.

The term “solvate” refers to a complex of variable stoichiometry (e.g., di-, tri-, tetra-, penta-, hexa-, and so on), which is formed by a solute (the hybrid compound) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The present invention further encompasses various crystalline forms (polymorphs) of the active agents described herein.

In cases where the active agent comprises one or more asymmetric centers, it can be utilized either as a purified stereoisomer or as a racemic mixture.

Derivatives and analogs of the active agents described herein are also encompassed by the present invention.

The term “derivative” describes a compound which has been subjected to a chemical modification while maintaining its main structural features. Such chemical modifications can include, for example, replacement of one or more substituents and/or one or more functional moieties, oxidation, reduction, and the like.

The term “analog” describes a compound that has structural features that are similar to that of the active agent and hence exhibits the same pharmacological activity.

Further preferably, each of the active agents described herein is provided as a pharmaceutical dosage unit, either a single unit or two units, as detailed herein.

Thus, preferably, the pharmaceutical composition comprises a pharmaceutical dosage unit of clomipramine. Preferably, the amount of the clomipramine ranges from about 15 mg to about 50 mg per dosage unit, most preferably from about 15 mg to about 30 mg per dosage unit.

Further preferably, the pharmaceutical composition comprises a pharmaceutical dosage unit of a PDE5 inhibitor as described herein. Preferably, the amount of tadalafil and vardenafil ranges from about 10 mg to about 20 mg per dosage unit and more preferably is about 10 mg; and the amount of sildenafil is about 25 mg per dosage unit.

The time required for clomipramine to reach peak plasma concentration is known to be about 2 hours longer than that required by tadalafil, and 3 hours longer than that required by either vardenafil or sildenafil. Therefore, in order to achieve overlapping efficacy windows of clomipramine and tadalafil, either as a unitary dosage form, or in separate dosage forms, release of tadalafil is preferably delayed by about 2 hours relative to release of clomipramine. In order to achieve overlapping efficacy windows of clomipramine and vardenafil or sildenafil, either as a unitary dosage form, or in separate dosage forms, and release of vardenafil or sildenafil is preferably delayed by about 3 hours relative to release of clomipramine.

The pharmaceutical composition preferably comprises, in addition to the erection-enhancing agent and the ejaculation-delaying agent, a pharmaceutically acceptable carrier, and may optionally further comprise one or more components selected from binding agents, stabilizers, diluents, excipients, surfactants, flavors, and odorants.

Herein, the phrase “pharmaceutically acceptable carrier” refers to a carrier or a diluent that does not cause significant irritation to a subject and does not abrogate the pharmacological activity and properties of the administered compounds.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections. Preferably, the compositions are formulated for oral administration.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

For oral administration, the active ingredients can be formulated readily by combining the active ingredients with pharmaceutically acceptable carriers well known in the art. Such carriers enable the active ingredients of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores.

Suitable excipients include, without limitation, fillers or diluents, disintegrating agents, lubricants, surfactants, and preservatives.

Non-limiting examples of fillers or diluents include dicalcium phosphate dihydrate; calcium sulfate; kaolin; silicon dioxide, titanium oxide; alumina; talc; sugars, including lactose, lactose monohydrate, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, microcrystalline cellulose, sodium carbomethylcellulose; and/or physiologically acceptable polymers such as polyvinylpyrrolidone (PVP) or croscarmellose sodium.

Suitable disintegrating agents include, without limitation, cross-linked polyvinyl pyrrolidone, agar, alginic acid or a salt thereof such as sodium alginate, or sodium starch glycolate

Non-limiting examples of lubricants include magnesium stearate, calcium stearate, stearic acid, glyceryl behenate, polyethylene glycol, and mixtures thereof;

Examples of surfactants include, but are not limited to, long alkyl chain sulfonates and alkyl aryl sulfonates such as sodium dodecylbenzene sulfonate; dialkyl sodium sulfosuccinates, such as sodium bis-(2-ethylhexyl)-sulfosuccinate; and alkyl sulfates such as sodium lauryl sulfate.

Suitable preservatives include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, propylparaben, mineral oil, oleic acid, olive oil, polyethers, polyoxypropylene butyl ether, polyoxypropylene cetyl ether, potassium sorbate, silicone oils, sodium propionate, sodium benzoate, sodium bisulfite, sorbic acid, stearic fatty acid, vitamin E, vitamin E acetate and derivatives, esters, salts and mixtures thereof.

The pharmaceutical composition may optionally be provided in any form suitable for oral administration, including, for example, a tablet, a pill, a dragee, a capsule, a liquid, a paste, a pellet, a syrup, a slurry, or a suspension. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

For example, tablets may be formed by direct compression of a powdered, crystalline or granular drug-containing composition, alone or in combination with diluents, binders, lubricants, disintegrants, colorants or the like. As an alternative to direct compression, compressed tablets can be prepared using wet-granulation or dry-granulation processes. Tablets may also be molded rather than compressed, starting with a moist material containing a suitable water-soluble lubricant. Drug-containing particles or beads are also prepared using conventional means, typically from a fluid dispersion. Further techniques for composition and administration of active ingredients may be found in “Remington's Pharmaceutical Sciences,” Mack Publishing Co., Easton, Pa., latest edition, which is incorporated herein by reference as if fully set forth herein.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising the erection-enhancing agent and the ejaculation-delaying agent of the present invention, formulated in a compatible pharmaceutical carrier may also be prepared, placed in an appropriate container, and labeled for treatment of an indicated condition, as is detailed herein.

As is mentioned hereinabove, the above described pharmaceutical compositions can be utilized in methods of enhancing sexual satisfaction or treating sexual dysfunction.

Such methods can be used to enhance sexual function of a healthy subject by enhancing erection or delaying the latency time to ejaculation or to treat a subject suffering from a sexual dysfunction, such as erectile dysfunction or premature ejaculation, or a combination thereof.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The sexual dysfunction, erectile dysfunction or ejaculatory dysfunction may be due to use of a drug, such as, for example, alcohol, nicotine, a narcotic, a stimulant, an antihypertensive, an antihistamine, and a psychotherapeutic agent. Alternatively, the dysfunction may be due to a medical condition, such as, for example, a back injury, an enlarged prostate gland, a circulatory problem, nerve damage, diabetic neuropathy, multiple sclerosis, a tumor, heart failure, lung failure, an endocrine disorder (such as a thyroid disorder, a pituitary disorder and an adrenal gland disorder), a hormone deficiency (such as low testosterone, low estrogen and low androgen), and a birth defect.

As discussed hereinabove, an association is frequently found between erectile dysfunction and premature ejaculation. For example, the psychological stress of maintaining an erection in a subject suffering from erectile dysfunction may lead to problems with control of ejaculation. Similarly, the stress of controlling ejaculation in a subject suffering from premature ejaculation may lead to inability to achieve erection. Furthermore, an increased incidence of erectile dysfunction has been reported in subjects receiving ejaculation-delaying agents for treatment of premature ejaculation.

The compositions described herein may further be beneficial in the treatment of erectile dysfunction caused by treatment with an ejaculation-delaying agent, and treatment of premature ejaculation caused by treatment with an erection-enhancing agent.

The present invention is therefore further of use of an erection-enhancing agent and an ejaculation-delaying agent, as described herein, for the preparation of medicaments for treating the conditions described herein.

The present invention is therefore further of methods which are effected by co-administration of an erection-enhancing agent and an ejaculation-delaying agent together as a multi-dosage form composition or as part of the same, unitary dosage form. “Co-administration” also includes administering a delayed release erection-enhancing agent and an ejaculation-delaying agent separately but as part of the same therapeutic treatment program or regimen. The components need not necessarily be administered at essentially the same time, although they can if so desired. Thus “co-administration” includes, for example, administering an erection-enhancing agent and an ejaculation-delaying agent as separate dosages or dosage forms, but at the same time.

Thus, according to further aspects of the present invention there are provided a method of enhancing sexual satisfaction, and a method of treating erectile dysfunction, each being effected by co-administering to a subject a pharmaceutically effective amount of an erection-enhancing agent, as described herein, and a pharmaceutically effective amount of an ejaculation-delaying agent, as described herein.

The methods and uses described herein are effected such that an efficacy window of the erection-enhancing agent and an efficacy window of an ejaculation-delaying agent substantially overlap, as described hereinabove.

Alternatively, or in addition, the methods and uses described herein are effected such that the erection-enhancing agent and the ejaculation-delaying agent act in synergy, as detailed herein.

Hence, the present invention further provides a method of treatment of sexual dysfunction, which is effected by co-administering to a subject a pharmaceutically effective amount of an erection-enhancing agent and a pharmaceutically effective amount of an ejaculation-delaying agent. Co-administering these agents is preferably effected substantially simultaneously.

The present invention further provides a method of treating ejaculatory dysfunction caused by treatment with an agent for the treatment of erectile dysfunction, comprising substantially simultaneously co-administering a pharmaceutically effective amount of an erection-enhancing agent and a pharmaceutically effective amount of an ejaculation-delaying agent.

As discussed hereinabove, agents for the enhancement of erection and for the delay of ejaculation are known in the art. Such agents may be used for the treatment of erectile dysfunction and for the treatment of premature ejaculation, respectively.

However, since the known agents for enhancement of erection and delay or ejaculation involve drugs which reach maximum plasma concentrations at different times, known methods of co-administration require administration of the individual medications at separate times, which is highly inconvenient for the patient, thereby reducing patient compliance. The prior art does not teach or suggest a method of substantially simultaneous co-administration of such agents.

The methods of the present invention overcome the disadvantages of the prior art by utilizing an erection-enhancing agent and an ejaculation-delaying agent, such that the efficacy windows of these agents substantially overlap. This can be achieved, for example, by using formulations in which the release of the erection-enhancing agent is delayed relative to the release of the ejaculation-delaying agent, such that both agents reach a plasma peak concentration substantially simultaneously, providing at least 20 minutes of overlapping maximal efficacy.

The method of the present invention are further advantageously superior to the presently known methods, by utilizing lower unit dosages of the active agents, based on the synergistic effect exhibited by these agents.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

The purpose of the study was to investigate the effects of co-administration of Clomipramine and various PDE5 inhibitors on patients suffering from both premature ejaculation and erectile disfunction.

Patients suffering from ejaculation time equal to or lower than 2 minutes and/or unsatisfactory erectile dysfunction, subjectively rated on a scale of 100%, were selected for retrospective analysis.

Patients were diagnosed during a meeting and received a combination therapy of a commercial PDE5 inhibitor and clomipramine, taken on an “as need” basis. After 8 weeks, the patients' performance was rated.

Patients suffering from both erectile dysfunction and premature ejaculation were treated either with low-dose commercially available dosage units of clomipramine (25 mg per dosage unit) or with unique low doses of clomipramine (15 and 30 mg per dosage) and with low-dose commercially available dosage units of a commercial PDE5 inhibitor (10 mg of tadalafil or vardenafil; 25 mg of sildenafil).

The results are presented in Table 1 below and in FIGS. 2a and 2b and clearly show the potent effect of a combination therapy of clomipramine and a PDE5 inhibitor in simultaneously treating both premature ejaculation and erectile dysfunction.

In addition, the obtained results clearly indicate that although relatively low doses of both clomipramine and the PDE5 inhibitor were prescribed and used in most of the patients, a significant erection enhancement (e.g., from less than 50% to 100 %) was recorded in more than 90% of the participants.

Thus, for example, 100% of patients suffering from mild erectile dysfunction (e.g., 60% to 90% subjectively rated erection and treated with 10 mg of tadalafil or vardenafil in combination with clomipramine reported 100% erection at the end of the study (after 8 weeks). Over 83% of patients suffering from moderate erectile dysfunction (e.g., 30% to 60% subjectively rated erection) and treated with 10 mg of tadalafil or vardenafil in combination with clomipramine reported 100% erection at the end of the study (after 8 weeks). Over 88% of patients suffering from severe erectile dysfunction (e.g., 0% to 30% subjectively rated erection) and treated with 10 mg of tadalafil or vardenafil in combination with clomipramine reported 100% erection at the end of the study (after 8 weeks).

Such a high rate of success in erectile dysfunction therapy has never been observed hitherto with such low doses of commercially available PDE5 inhibitors, when used alone. Thus, for example, the highest success rate reported for erectile dysfunction monotherapy with a 10 mg dosage unit of tadalafil, measured as percents of subjects attaining normal erectile function at study end point, is 51.8% for mild ED, 41% for moderate ED and 25% for severe ED (see, [18-21]).

The obtained results therefore demonstrate a synergistic effect of the combined therapy utilized herein on erectile dysfunction. Such a synergistic effect allows a treatment regimen that utilizes significantly lower doses of at least the PDE5 inhibitor and optionally of both clomipramine and the PDE5 inhibitor, while achieving a high rate of treatment success. Evidently, such a treatment regime is highly advantageous as it reduces the risk of adverse side effects associated with clomipramine and/or PDE5 inhibitors treatment. In Table 1 below, “NC” denotes no complain; PE denotes premature ejaculation; ED denotes erectile dysfunction; x denotes positive diagnosis; and 0 denotes negative diagnosis.

The statistical analysis of the results presented in FIGS. 1a and 1b is presented in Tables 2 and 3 below, respectively.

TABLE 2

| Column1 | | Column2 | |
|---|---|---|---|
| Ejaculation time before treatment | | Ejaculation time after treatment | |
| Mean | 1.031034483 | Mean | 9.698275862 |
| Standard Error | 0.102412133 | Standard Error | 0.776813458 |
| Standard Deviation | 0.779947568 | Standard | 5.916035045 |
| | | Deviation | |
| Count | 58 | Count | 58 |
| Confidence Level | 0.205076757 | Confidence Level | 1.555542107 |
| (95.0%) | | (95.0%) | |

TABLE 3

| Column1 | | Column2 | |
|---|---|---|---|
| Erection satisfaction before treatment | | Erection satisfaction after treatment | |
| Mean | 38.87931034 | Mean | 96.03448276 |
| Standard Error | 4.599907781 | Standard Error | 1.43290463 |
| Standard Deviation | 35.03185397 | Standard | 10.91267655 |
| | | Deviation | |
| Count | 58 | Count | 58 |
| Confidence Level | 9.211156377 | Confidence Level | 2.869342006 |
| (95.0%) | | (95.0%) | |

TABLE 1

| | Ejaculation Time | | Erection (% | | | | | | | | |
|---|---|---|---|---|---|---|---|---|---|---|---|
| | (minutes) | | satisfaction) | | Treatment | | | | | | |
| Patient | before | after | before | after | Viagra ® | Levitra ® | Cialis ® | Clomipramine | Diagnosis | | AGE |
| ID | treatment | treatment | (%) | (%) | (mg) | (mg) | (mg) | (mg) | PE | ED | (years) |
| 5 | 2 | 10 | NC | NC | | 0 | 0 | 30 | x | 0 | 35 |
| 8 | 1 | 20 | NC | NC | | 0 | 0 | 15 | x | 0 | 29 |
| 11 | 0.5 | 15 | NC | NC | | 0 | 0 | 25 | x | 0 | 29 |
| 15 | 0.5 | 10 | NC | NC | | 0 | 0 | 30 | x | 0 | 31 |
| 38 | 2 | 10 | NC | NC | | 0 | 0 | 30 | x | 0 | 36 |
| 40 | 0.5 | 5 | 60 | 100 | | 0 | 10 | 30 | x | 0 | 43 |
| 65 | 3 | 15 | NC | NC | | 0 | 0 | 30 | x | 0 | 41 |
| 80 | 0 | 8 | NC | NC | | 0 | 0 | 30 | x | 0 | 25 |
| 94 | 0.15 | 10 | 90 | 100 | | 0 | 0 | 10 | x | 0 | 44 |
| 114 | 0.5 | 10 | 100 | 100 | | 0 | 0 | 15 | x | 0 | 53 |
| 143 | 0 | 7 | NC | NC | | 0 | 0 | 25 | x | 0 | 24 |
| 166 | 0.1 | 4 | | NC | | 0 | 0 | 30 | x | 0 | 62 |
| 186 | 2 | 5 | NC | NC | | 0 | 0 | 30 | x | 0 | 28 |
| 199 | 4 | 20 | NC | NC | | 0 | 0 | 25 | x | 0 | 25 |
| 226 | 0.5 | 20 | NC | NC | | 0 | 0 | 30 | x | 0 | 39 |
| 272 | 0.5 | 15 | NC | NC | | 0 | 0 | 30 | x | 0 | 31 |
| 316 | 0 | 0.25 | | | | 0 | 0 | 30 | x | 0 | 32 |
| 431 | 0.5 | 3 | | | | 0 | 0 | 30 | x | 0 | 27 |
| 459 | 0.15 | 4 | | | | 0 | 0 | 30 | x | 0 | 29 |
| 462 | 0.5 | 20 | | | | 0 | 0 | 25 | x | 0 | 20 |
| 24 | 2 | 10 | 20 | 100 | | 10 | 0 | 30 | x | x | 29 |
| 26 | 0.15 | 6 | 0 | 80 | | 10 | 0 | 30 | x | x | 63 |
| 35 | 0.1 | 8 | 0 | 50 | | 0 | 10 | 30 | x | x | 65 |
| 41 | 0.5 | 3 | 20 | 80 | | 0 | 20 | 30 | x | x | 43 |
| 53 | 2 | 10 | 50 | 100 | | 0 | 10 | 30 | x | x | 36 |
| 54 | 0.05 | 10 | 50 | 100 | | 0 | 10 | 30 | x | x | 29 |
| 55 | 0 | 10 | 50 | 100 | | 0 | 10 | 30 | x | x | 30 |
| 63 | 0.15 | 8 | 0 | 100 | | 0 | 20 | 30 | x | x | 59 |
| 72 | 1 | 4 | 0 | 100 | | 10 | 0 | 30 | x | x | 49 |
| 77 | 2 | 10 | 70 | 100 | | 0 | 20 | 30 | x | x | 65 |
| 89 | 0.5 | 4 | 80 | 100 | | 0 | 20 | 30 | x | x | 57 |
| 90 | 0 | 4 | 0 | 60 | | 20 | 0 | 30 | x | x | 50 |
| 95 | 0.1 | 20 | 0 | 100 | | 0 | 20 | 30 | x | x | 48 |
| 97 | 0.15 | 5 | 50 | 100 | | 0 | 20 | 30 | x | x | 38 |
| 99 | 0.3 | 15 | 0 | 100 | | 0 | 10(5) | 30 | x | x | 20 |
| 108 | 1 | 4 | 100 | 100 | | 0 | 0 | 15 | x | x | 58 |
| 109 | 2 | 7 | 0 | 100 | | 0 | 20 | 15 | x | x | 37 |
| 111 | 3 | 7 | 70 | 100 | | 0 | 10 | 30 | x | x | 50 |
| 124 | 0.25 | 8 | 75 | 100 | | 0 | 10 | 30 | x | x | 38 |
| 169 | 2 | 7 | 90 | 100 | | 0 | 0 | 25 | x | x | 65 |
| 178 | 0.5 | 4 | 80 | 100 | | 0 | 10 | 30 | x | x | 55 |
| 184 | 1 | 8 | 80 | 100 | | 0 | 10 | 25 | x | x | 21 |
| 187 | 0.05 | 3 | 50 | 80 | | 0 | 10 | 30 | x | x | 53 |
| 228 | 2 | 7 | 80 | 100 | | 0 | 10 | 30 | x | x | 32 |
| 263 | 2 | 2 | 0 | 60 | | 0 | 20 | 25 | x | x | 67 |
| 270 | 2 | 15 | 70 | 100 | | 0 | 10 | 30 | x | x | 24 |
| 286 | 0.5 | 10 | 0 | 100 | | 0 | 10 | 30 | x | x | 34 |
| 292 | 1 | 20 | 80 | 100 | | 0 | 10 | 30 | x | x | 30 |
| 295 | 2 | 15 | 0 | 100 | | 0 | 10 | 15 | x | x | 54 |
| 309 | 1 | 8 | 0 | 100 | | 0 | 10 | 30 | x | x | 29 |
| 313 | 2 | 10 | 50 | 100 | | 0 | 10 | 30 | x | x | 40 |
| 315 | 2 | 12 | 0 | 100 | | 0 | 10 | 30 | x | x | 29 |
| 320 | 1 | 6 | 0 | 100 | | 0 | 10 | 25 | x | x | 31 |
| 327 | 0.5 | 3 | 0 | 100 | | 0 | 10 | 25 | x | x | 34 |
| 337 | 1 | 10 | 80 | 100 | | 0 | 10 | 30 | x | x | 47 |
| 340 | 2 | 30 | 90 | 100 | | 0 | 10 | 30 | x | x | 40 |
| 344 | 0.2 | 20 | 0 | 100 | | 0 | 10 | 25 | x | x | 32 |
| 417 | 0.15 | 7 | 80 | 100 | | 0 | 10 | 30 | x | x | 43 |
| 438 | 0.5 | 4 | 50 | 100 | | 0 | 10 | 25 | x | x | 52 |
| 452 | 0 | 6 | 40 | 80 | | 0 | 10 | 25 | x | x | 58 |
| 455 | 1 | 10 | 60 | 100 | | 0 | 10 | 30 | x | x | 37 |
| 460 | 1 | 2.5 | 0 | 100 | | 0 | 10 | 30 | x | x | 42 |
| 466 | 0.5 | 15 | 0 | 100 | | 10 | 0 | 30 | x | x | 29 |
| 472 | 1 | 10 | 0 | 100 | | 0 | 10 | 30 | x | x | 38 |
| 476 | 2 | 5 | 0 | 100 | | 0 | 10 | 30 | x | x | 28 |
| 480 | 1 | 20 | 0 | 100 | | 0 | 10 | 30 | x | x | 43 |
| 503 | 1 | 15 | 80 | 100 | | 0 | 10 | 30 | x | x | 35 |
| 510 | 1 | 5 | 50 | 80 | | 0 | 20 | 30 | x | x | 61 |
| 513 | 2 | 20 | 60 | 100 | | 0 | 10 | 30 | x | x | 60 |
| 527 | 1 | 5 | 80 | 100 | | 10 | 0 | 30 | x | x | 41 |
| 538 | 0.5 | 5 | 0 | 100 | | 0 | 10 | 30 | x | x | 30 |
| 539 | 0.15 | 20 | 50 | 100 | | 0 | 10 | 15 | x | x | 50 |
| 540 | 2 | 5 | 50 | 100 | 25 | 0 | 0 | 12.5 | x | x | 26 |
| 548 | 1 | 10 | 60 | 100 | | 0 | 10 | 25 | x | x | 44 |

### Example 2

A Dosage Form for Administration of an Erection-Enhancing Agent and an Ejaculation-Delaying Agent

The active ingredients are co-formulating into a single tablet or capsule, upon mixing and/or milling with suitable excipients.

Thus, the components of a tablet for oral administration are clomipramine (15 mg or 30 mg), vardenafil (10 mg), and gelatin, magnesium stearate, methylparaben, propylparaben, silicon dioxide, sodium lauryl sulfate, starch and titanium dioxide as inactive ingredients.

### Example 3

A Pulsatile Release Dosage Form for Administration of an Erection-Enhancing Agent and an Ejaculation-Delaying Agent

The dosage form is prepared by formulation of two individual compressed tablets, each having a different release profile, followed by encapsulating the two tablets into a gelatin capsule and then closing and sealing the capsule. The components of the two tablets are as follows.
Tablet 1: Immediate release formulation comprising clomipramine (30 mg) as active ingredient; gelatin, magnesium stearate, methylparaben, propylparaben, silicon dioxide, sodium lauryl sulfate, starch and titanium dioxide as inactive ingredients.
Tablet 2: Delayed release formulation comprising tadalafil (10 mg) as active ingredient; lactose monohydrate, croscarmellose sodium, hydroxypropylcellulose, microcrystalline cellulose, sodium lauryl sulfate and magnesium stearate as inactive ingredients.
Tablet 1 is uncoated. Tablet 2 is coated with methacrylic acid copolymer (delayed release coating material), triethyl citrate and talc.

### Examples 4-25

Dosage Forms for Administration of an Erection-Enhancing Agent and an Ejaculation-Delaying Agent

Additional dosage forms are prepared by co-formulation of the active -o ingredients into a single dosage form, as described in Example 2 above or by formulation of two individual compressed tablets, each having a different release profile, followed by encapsulating the two tablets into a gelatin capsule and then closing and sealing the capsule, as described in Example 3 hereinabove. The inactive ingredients in each of the two tablets are as described in Examples 2 and 3 hereinabove. The active ingredients in each tablet are presented in Table 4 below.

TABLE 4

| | Tablet 1: | Tablet 2: |
|---|---|---|
| Example | Ejaculation-delaying agent | Erection-enhancing agent |
| 4 | 30 mg clomipramine | 20 mg tadalafil |
| 5 | 15 mg clomipramine | 10 mg tadalafil |
| 6 | 15 mg clomipramine | 20 mg tadalafil |
| 7 | 30 mg clomipramine | 10 mg vardenafil |
| 8 | 30 mg clomipramine | 20 mg vardenafil |
| 9 | 15 mg clomipramine | 10 mg vardenafil |
| 10 | 15 mg clomipramine | 20 mg vardenafil |
| 11 | 30 mg clomipramine | 25 mg sildenafil |
| 12 | 30 mg clomipramine | 50 mg sildenafil |
| 13 | 15 mg clomipramine | 25 mg sildenafil |
| 14 | 15 mg clomipramine | 50 mg sildenafil |
| 15 | 20 mg paroxetine | 10 mg tadalafil |
| 16 | 20 mg paroxetine | 10 mg tadalafil |
| 17 | 10 mg paroxetine | 20 mg tadalafil |
| 18 | 10 mg paroxetine | 20 mg tadalafil |
| 19 | 20 mg paroxetine | 10 mg vardenafil |
| 20 | 20 mg paroxetine | 20 mg vardenafil |
| 21 | 10 mg paroxetine | 10 mg vardenafil |
| 22 | 10 mg paroxetine | 20 mg vardenafil |
| 23 | 20 mg paroxetine | 25 mg sildenafil |
| 24 | 20 mg paroxetine | 50 mg sildenafil |
| 25 | 10 mg paroxetine | 25 mg sildenafil |
| 26 | 10 mg paroxetine | 50 mg sildenafil |

### Example 26

The formulation and method described in Examples 2 and 3 hereinabove is repeated, except that drug-containing beads are used in place of tablets. For a pulsatile release formulation a first fraction of beads is prepared by coating an inert support material such as lactose with the erection-enhancing agent which provides the first (immediate release) pulse. A second fraction of beads is prepared by coating immediate release beads with an amount of delayed release coating material sufficient to provide a drug release-free period of 2-3 hours. The two groups of beads may be encapsulated as in Example 2, or compressed, in the presence of a cushioning agent, into a single pulsatile release tablet.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES CITED BY NUMERALS

### Other References are Cited Within the Text

1. Linnoila, M., et al. Clin. Pharmacol. Ther. 32(2):208-11, 1982.
2. Evans L. E. J., et al. Prog. Neuro-Psychopharmacol. 4:293-302, 1980.
3. Balant-Gorgia, A. E., et al. Clin. Pharmacokinet. 20(6):447-62, 1991.
4. Hyttel, J. Prog. Neuro-Psychopharmacol. Biol. Psychiat. 6:277-95, 1982.
5. Prescriber Info, Pfizer.
6. Faigle, J. W. and Dieterle, W. J. Int. Med. Res. 1:281-90, 1973.
7. Traskman, L, et al. Clin. Pharmacol. Ther. 26(5):600-10, 1974.
8. Monod, G. J. Int. Med. 342:9, 1995.
9. Beaumont, G. J. Int. Med. Res. 1:469-472, 1973.
10. Dawling, S., et al. Grad. Med. J. 56(suppl1):115-6, 1980.
11. Jones, R. B. and Luscombe, D. K. Postgrad. Med. J. 52(suppl3):62-7, 1976.
12. Corte, L. D., et al. J. Pharm. Pharmacol. 45:825-9, 1993.
13. Salonia, A., et al. J. Urol. 168(6):2486-9, 2002.
14. Chia, S. Int. J. Androl. 25(5): 301-305, 2002.
15. Strassberg et al. Journal of Sex and Marital Therapy 25: 89-101, 1999.
16. McMahon C. G., Journal of Urology 159: 1935-1938, 1998.
17. Daniel Drai, “A la recherche du temps perdu: présentation d'une nouvelle méthode de prise charge de l'éjaculation précoce”, submitted for L'université Paris-Nord for obtaining sexology diploma (1998).
18. Rosen R. C. et al. Urology 49:822-830, 1997.
19. Cappelleri J. C. et al. Urology 56:477-481, 2000.
20. Brock G. B. et al. J. Urol 168:1332-1336, 2002.
21. Carson C. C. et al. Br J Urol Int 93(2):1276-1281, 2004.

1-48. (canceled)

49. A pharmaceutical composition comprising a pharmaceutically effective amount of an erection-enhancing agent and a pharmaceutically effective amount of an ejaculation-delaying agent, the composition being such that an efficacy window of said an erection-enhancing agent and an efficacy window of said ejaculation-delaying agent substantially overlap.

50. A pharmaceutical composition comprising a pharmaceutically effective amount of an erection-enhancing agent and a pharmaceutically effective amount of an ejaculation-delaying agent, the composition being such that said erection-enhancing agent and said ejaculation-delaying agent act in synergy.

51. The pharmaceutical composition of claim 50, wherein said pharmaceutically effective amount of said erection-enhancing agent equals to or is lower than 25 mg per dosage unit.

52. The pharmaceutical composition of claim 51, wherein said pharmaceutically effective amount of said ejaculation-delaying agent is lower than 50 mg per dosage unit.

53. A pharmaceutical composition comprising clomipramine, as an ejaculation-delaying agent, and a PDE5 inhibitor, as an erection enhancing agent.

54. The pharmaceutical composition of claim 49, being such that a maximal efficacy window of said erection-enhancing agent and a maximal efficacy window of said ejaculation-delaying agent overlap for at least 20 minutes.

55. The pharmaceutical composition of claim 49, being designed such that a plasma peak concentration of said ejaculation-delaying agent and a plasma peak concentration of said erection-enhancing agent occur substantially simultaneously.

56. The pharmaceutical composition of claim 49, wherein at least said erection-enhancing agent is provided in a delayed release form.

57. The pharmaceutical composition of claim 49, wherein said ejaculation-delaying agent is clomipramine.

58. The pharmaceutical composition of claim 49, wherein said erection-enhancing agent is selected from the group consisting of a phosphodiesterase type 5 inhibitor, arginine and human DNA.

59. The pharmaceutical composition of claim 53, wherein said erection-enhancing agent is a phosphodiesterase type 5 inhibitor selected from the group consisting of tadalafil, vardenafil and sildenafil.

60. The pharmaceutical composition of claim 59, being formulated as a pharmaceutical dosage unit.

61. The pharmaceutical composition of claim 60, wherein an amount of said clomipramine ranges from about 15 mg to about 30 mg per dosage unit.

62. The pharmaceutical composition of claim 60, wherein said erection-enhancing agent comprises tadalafil or vardenafil.

63. The pharmaceutical composition of claim 62, wherein an amount of said erection-enhancing agent ranges from about 10 mg to about 20 mg per dosage unit.

64. The pharmaceutical composition of claim 62, wherein an amount of said erection-enhancing agent is about 10 mg.

65. The pharmaceutical composition of claim 60, wherein said erection-enhancing agent comprises sildenafil.

66. The pharmaceutical composition of claim 65, wherein an amount of said sildenafil is about 25 mg per dosage unit.

67. The pharmaceutical composition of claim 49, being provided in a delivery form suitable for oral administration.

68. A method of enhancing sexual satisfaction, the method comprising co-administering a pharmaceutically effective amount of an erection-enhancing agent and a pharmaceutically effective amount of an ejaculation-delaying agent, such that an efficacy window of said erection-enhancing agent and an efficacy window of said ejaculation-delaying agent substantially overlap, thereby enhancing sexual dysfunction.

69. A method of enhancing sexual satisfaction, the method comprising co-administering a pharmaceutically effective amount of an erection-enhancing agent and a pharmaceutically effective amount of an ejaculation-delaying agent, said erection-enhancing agent and said ejaculation-delaying agent acting in synergy, thereby enhancing sexual dysfunction.

70. A method of treating erectile dysfunction, the method comprising co-administering to a subject in need thereof a pharmaceutically effective amount of an erection-enhancing agent and a pharmaceutically effective amount of an ejaculation-delaying agent, such that an efficacy window of said an erection-enhancing agent and an efficacy window of said ejaculation-delaying agent substantially overlap, thereby treating erectile dysfunction.

71. A method of treating erectile dysfunction, the method comprising co-administering to a subject in need thereof a pharmaceutically effective amount of an erection-enhancing agent and a pharmaceutically effective amount of an ejaculation-delaying agent, said erection-enhancing agent and said ejaculation-delaying agent acting in synergy, thereby treating erectile dysfunction.

72. A method of treating erectile dysfunction, the method comprising co-administering to a subject in need thereof a pharmaceutically effective amount of a phosphodiesterase type 5 inhibitor, as an erection-enhancing agent and a pharmaceutically effective amount of clomipramine, as an ejaculation-delaying agent, thereby treating erectile dysfunction.

73. A method of treating sexual dysfunction, the method comprising substantially simultaneously co-administering to a subject in need thereof a pharmaceutically effective amount of an erection-enhancing agent and a pharmaceutically effective amount of an ejaculation-delaying agent, such that an efficacy window of said an erection-enhancing agent and an efficacy window of said ejaculation-delaying agent substantially overlap.

74. A method of treating sexual dysfunction, the method comprising substantially simultaneously co-administering to a subject in need thereof a pharmaceutically effective amount of an erection-enhancing agent and a pharmaceutically effective amount of an ejaculation-delaying agent, said an erection-enhancing agent and said ejaculation-delaying agent acting in synergy, thereby treating sexual dysfunction.

75. A method of treating sexual dysfunction, the method comprising co-administering to a subject in need thereof a pharmaceutically effective amount of a phosphodiesterase type 5 inhibitor as an erection-enhancing agent and a pharmaceutically effective amount of clomipramine as an ejaculation-delaying agent.

76. The method of claim 75, wherein said co-administering is performed substantially simultaneously.

77. A method of treating ejaculatory dysfunction caused by treatment with an agent for the treatment of erectile dysfunction, the method comprising substantially simultaneously co-administering to a subject in need thereof a pharmaceutically effective amount of an erection-enhancing agent and a pharmaceutically effective amount of an ejaculation-delaying agent, such that an efficacy window of said an erection-enhancing agent and an efficacy window of said ejaculation-delaying agent substantially overlap.

78. The method of claim 70, wherein said erectile dysfunction is caused by treatment with an ejaculation-delaying agent.

79. The method of claim 69, wherein said pharmaceutically effective amount of said erection-enhancing agent equals to or is lower than 20 mg per dosage unit.

80. The method of claim 79, wherein said pharmaceutically effective amount of said ejaculation-delaying agent is lower than 50 mg per dosage unit.

81. The method of claim 68, wherein said ejaculation-delaying agent is clomipramine.

82. The method of claim 68, wherein said erection-enhancing agent is a phosphodiesterase type 5 inhibitor.

83. The method of claim 82, wherein said phosphodiesterase type 5 inhibitor is selected from the group consisting of tadalafil, vardenafil and sildenafil.

84. The method of claim 83, wherein said ejaculation-delaying agent is clomipramine.

85. The method of claim 68, wherein said administering is effected orally.

86. The method of claim 68, wherein said erection-enhancing agent and said ejaculation-delaying agent are provided as a single dosage unit.

87. The method of claim 68, wherein said erection-enhancing agent and said ejaculation-delaying agent are provided as separate dosage units.
